(19) United States
(12) Patent Application Publication
(10) Pub. No.: US 2003/0016781 A1
(43) Pub. Date: Jan. 23, 2003

(54) Method and apparatus for quantitative stereo radiographic image analysis

(76) Inventor: Wen C. Huang, Auburn, AL (US)

Correspondence Address: Wen C. Huang, 2076 S. Evergreen Drive, Auburn, AL, 36830, US

(21) Appl. No.: 09/859,624

(22) Filed: May 18, 2001

(51) Int. Cl. G21K 4/00; A61B 6/02; G03C 9/00

(52) U.S. Cl. 378/41; 378/42; 382/128; 382/132

(57) ABSTRACT

A quantitative radiographic method and apparatus of determining the depth of a selected feature inside a three-dimensional object from a stereoscopic pair of left and right radiographic images to be presented to the left eye and right eye, respectively, of an operator. The method includes the steps of (a) producing the pair of images on the same object at slightly different angles, (b) operating image display devices to present the two images, and (c) performing and measuring horizontal shifting motions of the two images and obtaining the coordinates (X_{GA},Y_{GA},Z_{GA}) of an internal feature A with respect to a marker G according to a specified procedure. The procedure begins with aligning the image points of the marker G with their respective reference lines. The two reference lines lie on or very close to the image plane. Preferably, the same procedure is followed again for a second marker. The next step involves aiming and aligning the image points of the internal feature with respect to their respective reference lines. These procedures are carried out to allow for more convenient and accurate measurements of various image parallax values, which are in turn used to precisely calculate the location of an internal feature image of interest, such as a structural defect.

### FIELD OF THE INVENTION

The present invention relates to improved stereo radiographic image analysis methods and apparatus and, more particularly, to methods and apparatus for stereoscopically displaying radiographic images and quantitatively evaluating the size and location of a feature or defect inside a three-dimensional object such as a structural component or a human body.

### BACKGROUND OF THE INVENTION

High-energy radiations such as X-rays, gamma rays and neutrons are commonly used for non-destructive evaluation (NDE) of the internal defects of an object or for examination of the anomalies inside a human body. Radiographic images for either industrial NDE or medical diagnostic applications can be obtained by radiography-on-film, fluoroscopy (including digital radiography or computed radiography), and computed tomography (CT) methods. Each method has its advantages and disadvantages for a specific application.

Film radiography involves producing a sharp, natural size, permanent image of the internal features (e.g. flaws or anomalies) in an object. Such an image is usually not difficult to interpret. However, film radiography is often relatively slow and expensive.

Fluoroscopy or radioscopy entails the conversion of X-ray intensities into light intensities by utilizing a fluorescent screen. By placing the screen in the X-ray beam behind the specimen, one can produce an image of the specimen on the screen. The high X-ray absorbing capability of selected materials could result in low brightness images and hence poor sensitivity. One method to improve the fluoroscopic performance is to use a closed-circuit television (CCTV) camera to transfer the image on the fluorescent conversion screen on to a display monitor, relying on the electronic circuitry to enhance the signal and produce a bright image. Another technique is to use an image intensifier tube to convert X-rays into photons, which are then picked up by an image sensor. Commonly used image sensors are tube type TV cameras such as isocon, vidicons, and solid state charge coupled device (CCD) cameras. Another type of image sensor is the linear diode array (LDA), which can digitize and store the image to be viewed on a TV monitor. The digitization of the television signal has allowed a computer to be built into the system, and this advancement has greatly improved the attainable image quality. This development has also made it possible to perform real-time radiography.

Both the conventional film radiography and fluoroscopy only provide a two-dimensional (2-D) view of an object. In industrial applications, a 2-D image does not give a NDE technician an adequate perspective view on the spatial distribution of multiple flaws in a structural component, nor does it allow the technician to determine the depth of a particular flaw. For medical uses, a 2-D image may not provide a diagnostician adequate information as to the extent of a particular disorder, such as the exact depth of a foreign object in a human body.

To overcome some of the drawbacks of 2-D radiography, the approach of tomography was developed. Computed tomography (CT) involves obtaining and stacking a sequence of images representing 2-D cross sections or “slices” of the object. The 2-D images are acquired by rotating a thin, fan shaped beam of X-ray about the long axis of the object. X-ray attenuation measurements are obtained from many different directions across each slice. The 2-D images are reconstructed from these data through a sophisticated mathematical convolution and back projection procedure. A major drawback of tomography is that a NDE technician or diagnostician must mentally “stack” an entire series of 2-D slices in order to infer the structure of a 3-D object. The interpretation of a series of stacked 2-D images by an observer requires a great deal of specialized knowledge and skill. Further, such an approach is extremely time consuming and is prone to inaccuracy. The market price of a CT system typically exceeds a million U.S. dollars and, therefore, only select large hospitals or highly specialized governmental or industrial facilities could afford to have a CT system. Clearly, a need exists to develop a more affordable stereography system for 3-D inspection of the internal structure of an object.

Three-dimensional (3-D) or stereoscopic viewing provides a means for showing actual, more understandable spatial relationships among various features or flaws inside a body. Stereoscopic radiology was first introduced near the turn of the century. Extensive patent and open literature can be found that describes the methods or apparatus for producing stereoscopic radiographs.

Most of the techniques that have been used to achieve the stereo effect is based on the theory of parallax. Specifically, an image recorded from the perspective of the right eye must be seen by the right eye while an image recorded from the perspective of the left eye must be seen by the left eye. A simple way to accomplish this is to provide distinct and separate optical paths to each eye from each recorded image. For instance, the right and left eye image pairs may be recorded as transparencies which, when inserted in a common hand-held 3-D viewer, are presented to each eye separately through magnifying lenses. A second example using the principle of distinct and separate optical paths is the mirror based viewer system. In this system, the image pairs are positioned under a viewer which, through two pairs of angled mirrors, directs each image to its corresponding observing eye. These conventional 3-D viewers, normally without proper markers or references, do provide the observer a 3-D perspective. However, they do not readily permit determination of the specific depths in which certain features (or flaws) are located relative to a predetermined reference.

Disclosed in U.S. Pat. No. 3,984,684 (1976) is a technique that allows both production of the stereo effect and measurements of the depth and size of one or more internal parts of an object. The technique entails successively directing the X-ray beams from an X-ray tube through the object, then through a parallax grating, and finally onto the film. The grating is mounted on the film support system. The object and the film support system together are translated in parallel paths laterally with respect to the beam path at different speeds. These speeds are such that the film and the object are maintained in congruent alignment with the X-ray tube. The grating moves slightly out of congruency causing the beam passing through the grating to slightly scan the film during the transverse. Also, the angle at which the object is exposed to radiation from the X-ray tube gradually changes. The film image contains a series of side-by-side variable aspect views or images of the object, corresponding in number to the number of slits in the grating. These images when viewed with a lenticular screen produce a 3-D perception. This technique requires the utilization of a complicated radiograph-taking system and a lenticular screen as described above. The stringent congruent alignment requirement has made this technique not readily adaptable to existing X-ray radiography apparatus.

Liu and co-workers (International Journal of Pressure Vessels & Piping, Vol.44, 1990, pp.353-364 and Vol.48, 1991, pp.331-341) have proposed a quantitative stereoscopic method which not only provides a 3-D perspective view of the internal features but permits convenient calculations of the coordinates (X,Y,Z) of one or more flaws inside an object. The method begins with taking a pair of radiograph films with the X-ray tube shifted laterally in a plane parallel to the film between the two exposures (while the object remains stationary). Alternatively, the same result can be achieved by shifting the object laterally while the X-ray source remains fixed. These radiograph films are then examined in a stereoscopic viewer. With a suitable marker placed on the specimen surface when the radiographic films are being exposed, the position of a defect image inside the specimen can be determined. Two reference wires were placed above the pair of radiographic films to help on the calculation of the parallax distance. The method proposed by Liu, et al. provides a sound basis upon which more effective stereoscopes for quantitative radiography can be designed. This method, however, has been limited to film radiography. The procedures were lengthy and complicated. What is clearly needed is an improved method, which is based on Liu's principle and the various positive attributes of fluoroscopy, for conducting quantitative stereo radiology. The present inventor and his co-worker have developed several methods and related apparatus for quantitative stereoscopic radiography (U.S. Pat. No. 6,118,843, issued Sep. 12, 2000 and U.S. Pat. No. 6,115,449, Sep. 5, 2000, both to Huang and Jang). Further studies have led to the present invention which includes improved, more user-friendly, and faster methods for analyzing a pair of stereo radiographic images. The improved method and apparatus differ from the earlier versions (the above-cited two patents) in several aspects:

(1). The present method involves preferably placing the two reference lines very close to the image plane (e.g., positioning the reference wires almost in physical contact with the underlying films) or exactly on the image plane (e.g., reference lines internally generated on a monitor and the two images are on the same plane). Such an arrangement makes it easier to aim the reference lines on the respective images and makes the measurement of the parallax distances more accurate.

(2). The present inventor has found that by allowing the left reference line to coincide with the left image point of a selected feature or marker and the right reference line to coincide with the corresponding right image point, regardless if the left image, the right image, or both being shifted, the relative shift distance between the two images could be used to calculate the parallax distance. This has made it possible to eliminate several steps that were required in the earlier methods.

(3). In such an arrangement, it becomes unnecessary to use a stereoscope to ensure that the two images are accurately positioned and orientated to provide a 3-D view of the images. With the conditions as set forth in the above (1) and (2) being met, the pair of images are automatically in perfect registry to provide a 3-D perspective. A stereoscope can still be used, however, to observe the spatial dispersion of various features or defects inside the 3-D object and to help identify desired features or defects whose coordinates can be measured with the present method.

(4) By using a pattern recognition program, once an image point of a feature or marker in one of the two images (say, left image) is identified and positioned to coincide with a reference line (the left reference line), the corresponding image point of this feature or marker on the other image (right image) can be automatically identified and positioned to coincide with the other reference line (the right reference line).

(5) The apparatus includes two secondary platforms, instead of one secondary platform supported by one primary platform. The two secondary platforms are capable of sliding on an independent and separate basis along a horizontal X-axis direction. Displacement-metering sensors are provided to directly measure the relative displacement between one secondary platform and the other. It is this relative displacement value that is needed to calculate the parallax value of a particular image point.

### OBJECTS OF THE INVENTION

The principal objects of the present invention are:

(1) to provide an improved method of stereoscopically displaying radiography images and to allow for more convenient and faster determination of the location of an internal feature such as a broken bone in a human body.

(2) to provide an improved method and apparatus for not only stereoscopic viewing of the internal defect dispersion of an object through radiographic films but also quantitative determination of the location of a defect inside an object.

(3) to provide an improved method and apparatus for stereoscopic viewing of radiographic images displayed on a TV screen or a computer monitor and for determining the location of an internal feature.

### SUMMARY OF THE INVENTION

The present invention provides methods for conducting quantitative stereoscopic radiography, including film, video, digital, and computed radiography. These methods include the improved version of the above-mentioned Liu's method of film-based stereo radiography and further improvements over our earlier methods. Particularly included are methods that involve integrating reference line-based approaches with the great electronic imaging capabilities commonly associated with video radiography, digital radiography, or computer radiography.

Specifically, in one preferred embodiment, a method is disclosed which involves displaying a pair of radiographic images on the corresponding right and left video display devices of a stereoscopic viewing system. The pair of images can be obtained by transferring (e.g., scanning or digitizing) the corresponding radiography transparencies (films or negatives) or opaque prints onto one cathode ray tube (CRT), or two separate CRT monitors by using a common image scanner or TV camera. Alternatively, the images can be obtained by directly using common fluoroscopy devices to display the images without going through the intermediate film-taking procedure. This can be accomplished by directing the beam of an X-ray source (or other types of high energy radiation) through an object and by using an image intensifier to convert the radiation into visible light, allowing the image to be shown on a fluorescent screen. Alternatively, the light photons emitted from the image intensifier may be recorded by an image sensor or reader which delivers the images either directly to video display devices (including computer monitors) or to an image storage device. In the latter case, the images will be later played back to the video display devices for examination.

As an example, referring to FIG. 1(A), both the right and left video display devices are each provided with a vertical reference line, which can be simply a thin opaque wire attached vertically (herein referred to as transversely, or in the Y-coordinate direction) to the display screen. The reference lines may be written onto the screen surface by using a marking pen or internally generated on a computer monitor. Proper movement means are provided to allow the two images to be shifted laterally (horizontally, in the X-coordinate direction) either simultaneously in congruency or with respect to each other. The X-axis also lies substantially parallel to the line segment connecting the two eyes of an operator. Displacement-metering devices are given to measure and record these shift distances. Shifting of the two images can be accomplished by positioning the two display devices on a slidable platform, hereinafter referred to as the primary platform, and then horizontally translating this platform. Either the left or the right display device is also supported on a secondary platform which is capable of moving horizontally, independent from the movement of the primary platform. Alternatively, both display devices can be supported on two separate secondary platforms. The secondary platform(s) is (are) slidably attached to the top surface of the primary platform. The movements of both secondary and primary platforms can be recorded by any movement-measuring means such as a micrometer, sliding caliper, optical encoder, linear slide, laser beam-based displacement sensor, linear variable differential transformer (LVDT), or any other type of displacement sensor. These measuring means are used to measure out the shift distances of both marker and defect images on one of the image pair relative to those on the other image.

It may be noted that, by referring to FIG. 1(A) again, the X-coordinate direction is the X-ray source shifting direction (when the radiography image is taken), which is also parallel to the platform movement direction. The transverse direction on the image plane is the Y-coordinate direction, which is the vertical direction in FIG. 1(A). The Z-coordinate direction is perpendicular to both the X-direction and Y-direction; i.e. being normal to the image plane and substantially in the sample depth direction.

In another embodiment, the pair of radiography images may be shown side by side on the same display unit, such as a TV monitor or a computer monitor. The monitor screen is artificially divided into two zones: a left zone showing the image to be presented to the left eye and a right zone showing the image to be presented to the right eye of an observer. Vertically across each zone is one of the afore-mentioned reference lines or wires. There exist commercially available image processing software-hardware packages that are capable of providing and measuring the concurrent and separate movements of the two images on a TV screen or computer monitor. In yet another embodiment, the monitor is mounted on a horizontally slidable primary platform, which provides simultaneous shifting of the two images. Shifting of one image with respect to the other can be executed on the monitor by a simple computer command.

The two images may be viewed by an optical observing unit (a stereoscope) which is composed of two optical paths, one for observing the left image by the left eye and the other for observing the right image by the right eye of an observer. Each optical path begins with an objective lens that is capable of seeing a broad image area and directing the image to a pair of angled mirrors or prisms. The mirrors or prisms in turn send the image through an eyepiece into one eye of the observer. The separation between the two eyepieces is adjustable to suit different observers. The separation between the two objective lenses is designed to be in accord with the dimensions of, and the separation between the two images to ensure a broad viewing field. This pair of optical paths preferably are provided with a vertical movement means which is in turn supported by a sturdy stand. This vertical movement provision permits the observer to cover a wider viewing area in cases the display screen is wider than the range covered by the pair of objective lenses when in one specific height. It may be noted that the present method does not require the utilization of a stereoscope, but it can be used advantageously to provide a stereo perspective of how one internal feature is spatially related to other features, particularly in the depth direction.

In summary, the present invention discloses improved methods for stereoscopically displaying radiographic images of the internal structure of an object and for determining the spatial coordinates of selected feature images inside the object. The method is composed of several steps:

(a) producing a pair of images on the same object taken from slightly different angles with image reference markers being placed near or on selected positions (preferably on or near the top or bottom surface) of the object when irradiated; (b) operating image display devices to present this pair of images with the two images being set up in a definitive orientation so that when the images are being viewed with both eyes by an observer, the two lines of sight connecting the eye balls and the corresponding image points of the image pair intersect; the two images being respectively provided with two stationary, transversely aligned reference lines across the image plane in the Y-direction; (c) performing and measuring horizontal shifting motions of the two images according to a sequence of procedures to be specified at a later section. These procedures basically involve aiming and aligning the image points of an internal feature with their respective reference lines. The same procedures are then repeated to align the image points of a marker with their respective reference lines. Preferably, the same procedures are followed again for a second marker. These procedures are carried out to allow for more convenient and accurate measurements of various image parallax values, which are in turn used to precisely calculate the location of an internal feature image of interest.

### BRIEF DESCRIPTION OF THE DRAWINGS

FIG. 1(A) Schematic showing the major components of a preferred apparatus for a stereoscopic radiograph observing and measuring apparatus; the apparatus including a slidable secondary platform 28 supported by a slidable primary platform 30 with both platforms being supported by a stationary base 42 or frame. (B) An apparatus similar to (A), but with two separate secondary platforms 28,29, which are capable of undergoing displacements with respect to each other and are supported by a base 42.

FIG. 2 Schematic showing the two optical paths in the observing compartment (a stereoscope).

FIG. 3(A) Geometrical relationships between a lead marker G, an internal defect A, and their images g₁, g₂ and a₁, a₂ on a radiographic film or image intensifier screen (referred to as image plane, p). An image is recorded (e.g., a radiograph p₁ is taken) when the X-ray source is located at S₁. A second image is recorded (e.g., a second radiograph p₂ is taken) when the source is at S₂. (B) The corresponding situation where the two images are taken sequentially; the second image is taken after the object is shifted laterally while keeping the X-ray source stationary.

FIG. 4 Geometrical relationships between two lead markers G, K, an internal defect A, and their respective images g₁, g₂, k₁, k₂ and a₁, a₂ on a radiographic film or an image intensifier screen (eventually on a computer monitor or video display screen). This diagram helps illustrate the derivation of the formulae used in depth calculations of internal defects.

FIG. 5 Geometrical relationships between the lead marker G, an internal defect A, and their respective images g₁, g₂, and a₁, a₂ on a radiographic film or an image intensifier screen (eventually on a computer monitor or video display screen). This diagram helps illustrate the derivation of the formulae used in the calculations of horizontal image shifts or the X-coordinate value of an internal defect position.

FIG. 6 Geometrical relationships between the lead marker G, an internal defect A, and their images g₁, g₂, and a₁, a₂ on a radiographic film or an image intensifier screen (eventually on a computer monitor or video display screen). This diagram helps illustrate the derivation of the formulae used in the calculations of transverse image shifts or the Y-coordinate value of an internal defect position.

FIG. 7 Schematic showing the procedure to follow for measuring and calculating the depth of a defect.

FIG. 8 A block diagram illustrating the major components and steps involved in the production and display of image pairs on video display devices.

### DETAILED DESCRIPTION OF THE PREFERRED EMBODIMENTS

A detailed description of preferred embodiments of the present invention are disclosed herein. The described embodiments are to be understood as merely exemplary of the invention, which may be embodied in various forms. Therefore, specific structural and functional details disclosed herein are not to be construed as limiting, but merely as a basis for the claims and as a representative basis for teaching those who are skilled in the art to variously employ the present invention for a wide range of appropriately detailed structures.

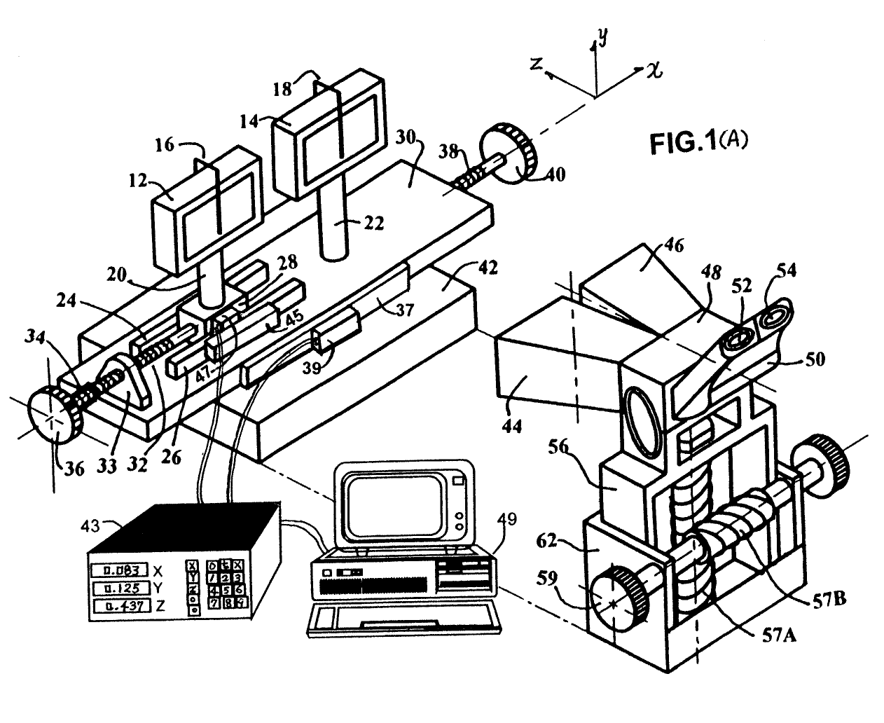

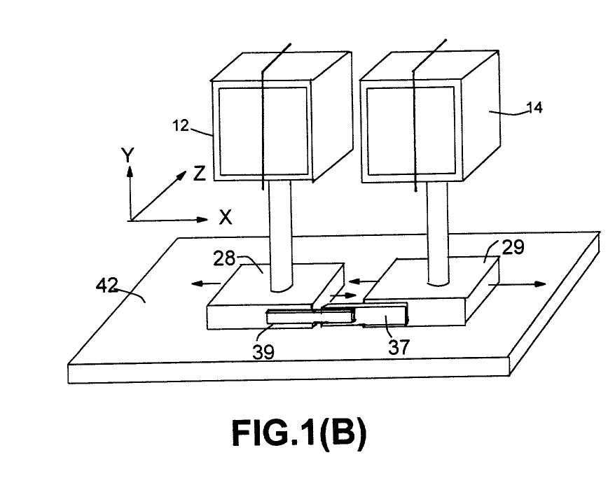

FIG. 1(A) schematically shows the major components of a preferred design for a stereoscopic radiograph observing and measuring apparatus that can be used to carry out the procedures specified in the presently invented method. Two video display devices 12, 14 are used to display a pair of radiographic images. Two reference lines 16, 18 are provided across the respective screens of the two display devices. These two reference lines may be two thin opaque wires located in front of, but very close, to the screen plane. These wires may be physically held in place by fastening means (not shown) on the apparatus base 42. These wires are not allowed to move along with the display devices 12,14 and will provide the necessary position references for measuring the image shifts and defect locations (to be explained later).

Both display devices are supported by a slidable platform 30, referred to as the primary platform, through their respective stands, 20 and 22. One of the two video display devices (shown to be the left one 12 in FIG. 1(A), but could have been the right one 14), through its stand 20, is positioned on a slidable platform 28, referred to as the secondary platform. The stand 20 is preferably fastened to or integrated with platform 28. Also, the stand 22 is preferably fastened to or integrated with platform 30. Platform 28 is allowed to slide horizontally between two guiding posts 24, 26 forming a trough to slidably accommodate platform 28. The sliding movement of platform 28 may be driven by any drive means. Shown in FIG. 1(A) is a simple driving mechanism that is constituted by a threaded shaft 32, supported by a shaft housing 33, a micrometer 34, and a turning handle 36. By turning the handle 36, one can advance or retreat the shaft screw 32 to drive the secondary platform 28 horizontally. The motion of the shaft may be either manually driven (e.g., by spinning the handle to a desired number of turns) or driven by any power tool (e.g., an electrical motor, hydraulic piston, pneumatic, solenoid, or other types of actuators). What is schematically shown in the left portion of FIG. 1(A) represents one of the many common sliding mechanisms that can be utilized to generate reversible sliding motions for a part. Those who are skilled in mechanical art may select from a wide array of sliding mechanisms that are commonly used and are mostly commercially available. For example, those worm shaft-worm gear combinations commonly used in moving the platforms of a milling machine or a lathe may be used for moving the secondary platform and measuring its travel distance. Similarly, a drive means, represented by 38,40 is also provided for the primary platform 30, to move the two images simultaneously. A displacement measuring means, such as a micrometer, is provided for this primary platform. The secondary platform 28 is used to horizontally shift one image with respect to the other. The two drive mechanisms need not be of same type or dimensions. The complete assembly is supported by a sturdy base 42.

Alternatively, each display device may be provided with a separate secondary platform. As shown in FIG. 1(B), two separate secondary platforms 28,29 are both supported on a stationary base 42. The two secondary platforms are capable of sliding horizontally along the X-axis direction of an X-Y-Z coordinate system indicated in FIG. 1(B). The relative separation of these two secondary platforms can be measured by any displacement-metering means. For instance, a set of optical encoder represented by 37,39 are attached to 29 and 28, respectively. When one platform is shifted relative to the other, the encoder picks up the displacement signals, which may be acquired and displayed by a digital display unit and/or computer. Advantageously, additional two micrometers or sliding calipers may be respectively attached to the two secondary platforms. The difference in readings shown on these two micrometers would indicate the relative displacement between the two display units or between the two images thereon.

In FIG. 1(A) and 1(B), the micrometers are connected in-line to measure the sliding distances of the two platforms (two secondary platforms or one primary plus one secondary platform). Again, there are many simple ways of measuring the travel distance of a part. One may choose to use an optical encoder, a laser beam, a linear slide (commonly used in a CNC mill), or just a simple sliding caliper, etc. In FIG. 1(A), two sets of optical encoder, 37,39 and 45,47, are used to acquire the displacement signals, which are displayed by a digital display unit 43 and/or computer 45. To use any other type of drive means or travel measuring means in the present context would merely represent a simple variation of the present invention. In a further preferred embodiment, the micrometer may be replaced by or supplemented with a displacement sensor that is capable of converting the mechanical displacement data into electrical signals in analog or digital form. These sensors are very commonly used in the field of physical measurements. Examples include the linear variable differential transformer (LVDT) or an extensometer-type sensor commonly used in the mechanical testing of materials. Preferably, the analog signals are further converted into digital signals through an analog-to-digital (AD) converter means. These digital signals then are directly displayed in a digital display means such as a liquid crystal display. These signals may also be further used by a computer to calculate the acquired image shift distances and the spacial coordinates (X,Y,Z) of an internal feature of an object.

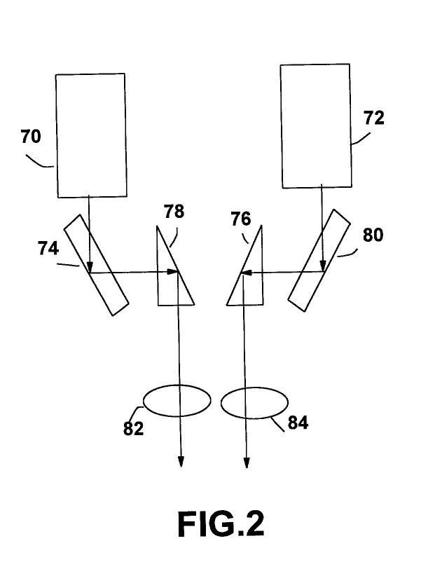

The two images shown on the screens of display devices 12,14 are to be viewed by the observing unit, shown on the right lower portion of FIG. 1(A) or that of FIG. 1(B). Housed in casings 44,46,48 are mirrors and lenses that are required to direct the light from the two images to an adjustable binocular 50 including two eyepieces 52,54. This optical assembly, 44 through 54, provides two distinct and separate optical paths to meet the parallax requirement of generating a stereo perception; i.e. an image recorded from the perspective of the right eye now can be seen by the right eye while an image recorded from the perspective of the left eye seen by the left eye. The arrangement of the two optical paths is schematically shown in FIG. 2, in which the two images 70,72 are respectively reflected and re-directed through mirrors or prisms 74,78 and 76,80, and then through the lenses 82,84 in eyepieces 52,54 into the left and right eye of an observer. Such an optical path assembly device is essentially a mirror stereoscope commonly used in viewing geological survey maps.

The optical path assembly device is supported by a stand 56, which preferably has a height-adjusting means (not shown) to move the assembly up and down as desired. Any releasable fastening means with sliding provisions, any proper ball bearing-screw combination or chain-wheel combination possibly driven by a motor means, can be set up to drive the optical assembly up and down. The stand 56 is connected to or integrated with a sturdy base 62, which can be connected to or integrated with the base 42 of the two platforms. Such an optical path assembly device may also be directly attached to one or two sides of a computer monitor or video display device (not shown).

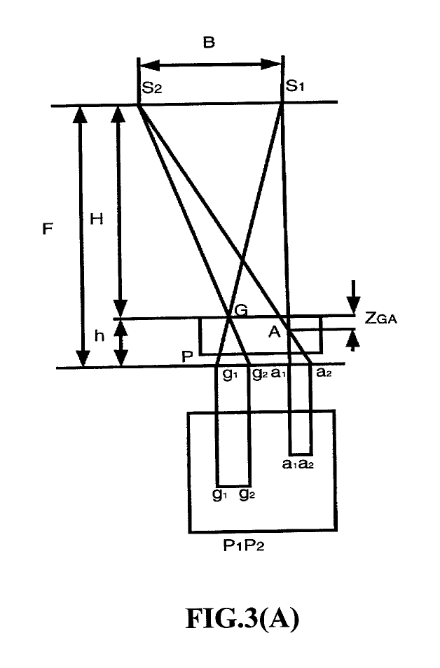

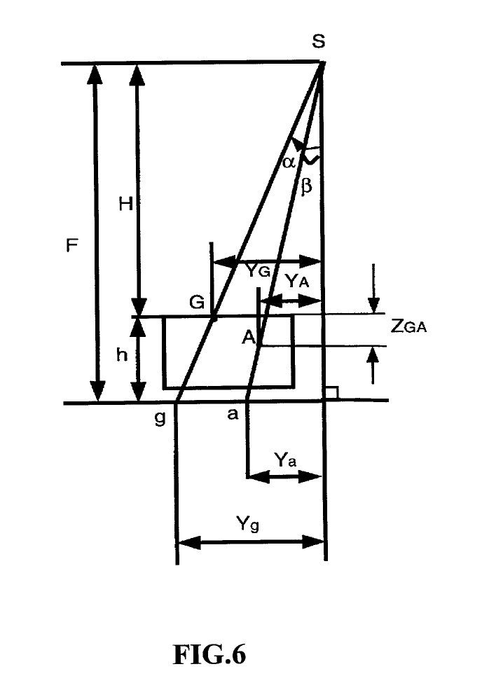

The operating principles for the presently invented quantitative stereoscopic radiography apparatus may be best illustrated by referring to FIGS. 3-7. Prior to taking radiographs or generating X-ray images on an image intensifier (or image sensor and reader), the image orientation must be defined and reference markers established. Reference markers are set up to meet specific measurement needs. For example, in order to measure the vertical depth from the top surface of an object to an internal flaw, a small-sized lead marker may be placed on the top surface of the object. This reference marker may be selected to be any surface or internal feature of the object with a known position. The basic procedures for carrying out radiography are shown in FIG. 3(A). An imaging plate P (either a radiographic film or an image intensifying device) is placed behind the object. An image is produced on plate P₁ at a focal length F with the radiation source located at S₁. On this image plate P₁ are shown the image point g₁ of a reference marker G and the image point a₁ of a flaw A. The radiation source is then shifted laterally by a distance B to a new position S₂ while the object remains stationary. A second image is then produced on plate P₂ with a focal length F. This plate P₂ now contains the image point g₂ of G and the image point a₂ of A. Alternatively, one may choose to maintain the radiation source stationary while shifting the object laterally by a distance B (FIG. 3(B)). With all other parameters maintained constant, both modes of image acquisition will yield the same results.

Referring to FIG. 3(A), the depth from the reference marker G to flaw point A may be derived as follows: Let Z_{GA} be the vertical distance from point G to point A, h the distance from the top surface of the object to the imaging plate, then H=F−h. (Related mathematical symbols are herein defined: ˜ means “being similar between two triangles”; ∵ means “because”; ∴ means “therefore”; Δ, when followed by three letters, denotes a triangle; a₁a₂ means the distance between a₁ and a₂)

$$\begin{matrix}∵ Δ {S}_{1} A {S}_{2} \sim Δ {a}_{1} A {a}_{2} & \\ ∴ \frac{{a}_{1} {a}_{2}}{B} = \frac{h - {Z}_{G A}}{H + {Z}_{G A}} & \\ T h e n {Z}_{G A} = \frac{B \cdot h - {a}_{1} {a}_{2} \cdot h}{B + {a}_{1} {a}_{2}} & ( a ) \\ ∵ Δ {S}_{1} G {S}_{2} \sim Δ {g}_{1} G {g}_{2} & \\ ∴ \frac{{g}_{1} {g}_{2}}{B} = \frac{h}{H} & \\ T h e n h = \frac{{g}_{1} {g}_{2} \cdot H}{B} & ( b )\end{matrix}$$

Substitution of (b) into (a) gives

$${Z}_{G A} = \frac{( {g}_{1} {g}_{2} - {a}_{1} {a}_{2} ) H}{B + {a}_{1} {a}_{2}} = \frac{H}{B} ( {g}_{1} {g}_{2} - {a}_{1} {a}_{2} ) \cdot {( 1 + \frac{{a}_{1} {a}_{2}}{B} )}^{- 1} \qquad ( c )$$

In a normal radiographic image taking situation, Z_{GA}<<H, hence a₁a₂<<B; therefore, Eq.(c) may be simplified as:

$${Z}_{G A} = \frac{H}{B} ( {g}_{1} {g}_{2} - {a}_{1} {a}_{2} ) \qquad ( d )$$

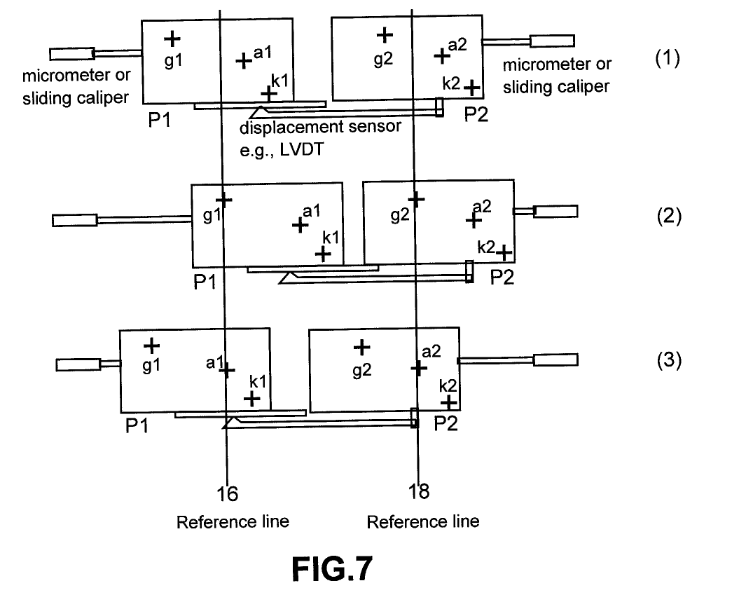

In Eq.(d), H and B can be determined during the image taking step, (g₁g₂−a₁a₂) can be measured by examining the images on plates P₁ and P₂. Therefore, Z_{GA} can be readily calculated provided that the apparatus permits determination of (g₁g₂−a₁a₂). The detailed procedure for determining (g₁g₂−a₁a₂) is given as follows (see FIG. 7):

Step 1: Place the images of plates P₁ and P₂ in a correct orientation according to the directional marks of the plate. The two images must be parallel to each other side by side.

Step 2: Gently shift the primary platform 30 and the secondary platform 28 (referring to FIG. 1(A)) or shift the two secondary platforms (referring to FIG. 1(B)), sequentially or concurrently, to insure that the left reference line coincides with the left image point g₁ and the right reference line coincides with the right image point g₂. At this moment of time the relative shift distance between the two films (or the two digital images or video images), specified by P_{G,} may be read off from one micrometer (FIG. 1(A)) or two micrometers if there are two secondary platforms FIG. 2(B)). A displacement sensor, such as a LVDT mounted between the two display devices, may be used to directly measure out the relative displacement. The P_{G} values may be automatically computed by a computer if the displacement signals are digitally transferred into the computer. If the shifting of the images is conducted directly on a computer monitor by using a mouse, the relative shift distance between the two images can also be automatically calculated by, for instance, counting the number of pixels traversed by such a shifting.

Step 3: Follow a similar procedure to move the platforms to bring image a₂ to fall on the right reference line 18 and to bring image a₁ to fall on left reference line 16. Then, record the relative travel distance P_{A} of the two platforms. Here, P_{G}−P_{A}=ΔP_{GA}=(g₁g₂−a₁a₂).

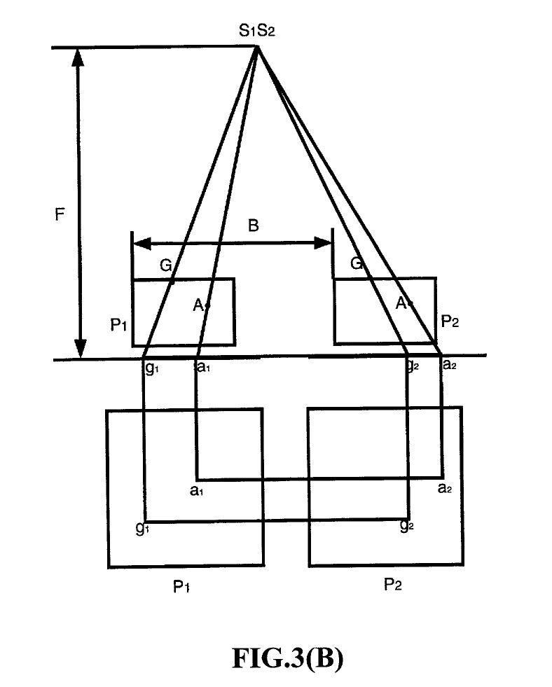

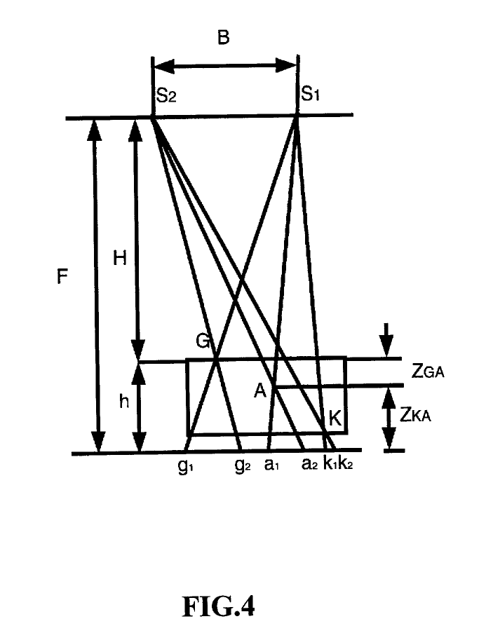

In actual radiography practice, the focal length F may not be accurately measurable, resulting in some inaccuracy in defining H=F−h. Consequently, there may be a large error with Z_{GA}=H/BΔP_{GA} In order to overcome this potential problem, one may set up another lead marker K preferably at the bottom surface of the object. Based on FIG. 4, another depth equation for Z_{GA} may be derived as follows: A simple manipulation of Eq.(b) leads to H=Bh/g₁g₂ which, upon substitution into Eq.(d), gives

$${Z}_{G A} = \frac{h}{{g}_{1} {g}_{2}} ( {g}_{1} {g}_{2} - {a}_{1} {a}_{2} ) = h ( 1 - \frac{{a}_{1} {a}_{2}}{{g}_{1} {g}_{2}} ) \\ ∵ {a}_{1} {a}_{2} = K {a}_{1} - K {a}_{2} ; {g}_{1} {g}_{2} = K {g}_{1} - K {g}_{2} ; \\ ∴ {Z}_{G A} = h ( 1 - \frac{K {a}_{1} - K {a}_{2}}{K {g}_{1} - K {g}_{2}} )$$

$$L e t \text{:} K {a}_{1} - K {a}_{2} = Δ {P}_{K A} ; K {g}_{1} - K {g}_{2} = Δ {P}_{K G}$$

$$T h e n \text{:} {Z}_{G A} = h ( 1 - \frac{Δ {P}_{K A}}{Δ {P}_{K G}} )$$

Here, h is a parameter (the separation between the top surface of the object and the imaging plate) that can be measured accurately. Further, ΔP_{KA} and ΔP_{KG} are parameters that can be measured by the presently proposed apparatus. Their measurement procedures are similar to those for ΔP_{GA} (Step 4).

Step 4: Referring to FIG. 7 again and follow a procedure similar to Step 2 or 3. Move the platforms to bring image k₂ to fall on the right reference line 18 and to bring image k₁ to fall on left reference line 16. Then, record the relative travel distance P_{K} of the two platforms. Here, P_{G}−P_{K}=ΔP_{GK}=(g₁g₂−k₁k₂) and P_{K}−P_{A}=ΔPₖ =(k₁k₂−a₁a₂). Utilization of the above equations can significantly improve the accuracy for Z_{GA}.

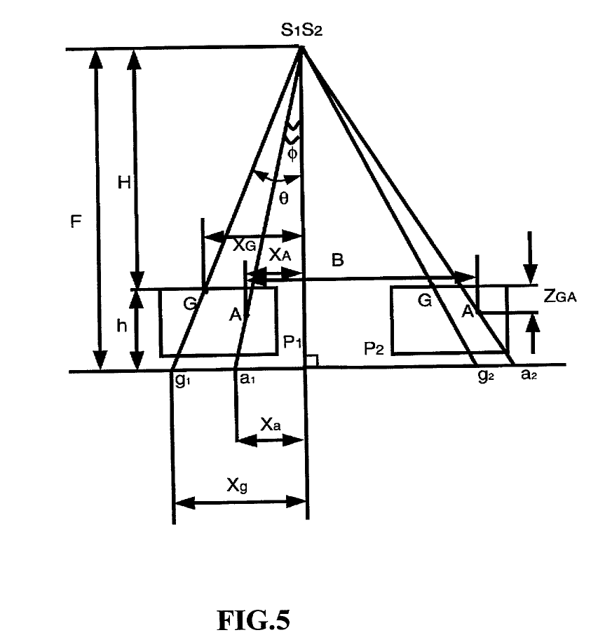

Based on FIG. 5, the horizontal coordinate from flaw point A to reference marker point G can be derived as follows: Draw a vertical line from the radiation source S₁,S₂ to the plate P. Let X_{GA}=the horizontal distance from point G to point A; X_{A}=the distance from point A to the vertical line; X_{G}=the distance from point G to the vertical line; Xₐ=the distance from point a₁ to the vertical line; X_{g}=the distance from point g₁ to the vertical line. Then,

$$∵ tan ϑ = \frac{{X}_{G}}{H} = \frac{{X}_{g}}{F} \\ ∴ {X}_{G} = \frac{{X}_{g} \cdot H}{F} ; {X}_{g} = \frac{{X}_{G} \cdot F}{H} \\ ∵ tan φ = \frac{{X}_{A}}{H + {Z}_{G A}} = \frac{{X}_{a}}{F} \\ ∴ {X}_{A} = \frac{{X}_{a} ( H + {Z}_{G A} )}{F} : {X}_{a} = \frac{F \cdot {X}_{A}}{H + {Z}_{G A}}$$

Also, let ΔXag be the horizontal distance from the image point g₁ to image point a₁, then ΔXag=Xg−Xa. Substitution of the expressions for Xa and Xg into this equation leads to:

$$Δ {X}_{g a} = \frac{F \cdot {X}_{G}}{H} - \frac{F \cdot {X}_{A}}{H + {Z}_{G A}} \\ ∴ {X}_{A} = \frac{( H + {Z}_{G A} ) ( F \cdot {X}_{G} - H \cdot Δ {X}_{g a} )}{F \cdot H}$$

Since X_{GA}=X_{G}−X_{A} and if the condition of X_{G}=B/2 can be met during the radiography imaging step, then X_{GA} can be expressed as:

$${X}_{G A} = \frac{B}{2} - \frac{( H + {Z}_{G A} ) ( \frac{F \cdot B}{2} - H \cdot Δ {X}_{g a} )}{F \cdot H}$$

where ΔXga is an unknown variable; however, it may be determined by examination of the image from P₁ with a transversely aligned ruler on the apparatus (or by simply moving a cursor on a computer monitor in the case of digital images). Then, by plugging ΔXga into the equation for X_{GA}, one obtains the value of X_{GA}.

By following similar procedures, the longitudinal distance Y_{GA} from the reference point G to flaw point A may be derived as follows:

$${Y}_{A} = \frac{F \cdot {Y}_{G} ( H + {Z}_{G A} ) - H ( H + {Z}_{G A} ) \cdot Δ {Y}_{g a}}{H \cdot F}$$

Deducting from both sides of the equation by the same amount Y_{G}, one obtains

$${Y}_{G A} = \frac{Δ {Y}_{g a} ( H + {Z}_{G A} )}{F} - \frac{{Y}_{G} \cdot {Z}_{G A}}{H}$$

In real practice, Z_{GA}<<H, therefore,

$${Y}_{G A} = \frac{Δ {Y}_{g a} ( H + {Z}_{G A} )}{F} .$$

With the present radiography apparatus, one can use a transversely aligned ruler to measure ΔYga directly on the film P₁ or P₂ and, therefore, readily obtain the value of Y_{GA}. In the case of digital image analysis, the value of ΔYga may be readily obtained by moving a cursor.

In the equations for X_{GA} and Y_{GA}, F and H can not be accurately measured. In order to avoid the potential error, one may obtain the values of F and H through further calculations. Referring to FIG. 4 again:

$$∵ Δ {S}_{1} G {S}_{2} \sim Δ {g}_{1} G {g}_{2} \\ ∴ \frac{H}{B} = \frac{h}{{g}_{1} {g}_{2}} \\ ∵ {g}_{1} {g}_{2} = k {g}_{1} - K {g}_{2} = Δ {P}_{G K} \\ ∴ H = \frac{h}{Δ {P}_{G K}} \cdot B ; F = H + h = h ( 1 + \frac{B}{Δ {P}_{G K}} )$$

In the above equations, ΔP_{GK} can be accurately measured by the proposed apparatus, the measurement method being the same as that for ΔP_{GA} described earlier.

When viewing an object with both eyes, one sees different sides of the object from two different directions. Therefore, if a proper pair of perspective drawings, photos or other type of images corresponding to these two sides of the object are separately provided in front of their respective eyes, then the images on the retinas will provide a perception identical to what would have been visioned with both eyes. A 3-D optical model in space is thus sensed or perceived. This stereoscopic vision, obtained from viewing the preserved images, may be termed reproduction of the stereoscopic effect. The drawings, photos or images of other form producing such an effect may be termed a “photo-couple”. This kind of observation with a stereoscopic effect is herein referred to as stereoscopic observation.

The above-described principle of stereoscopic observation suggests that the following conditions must be fulfilled in order to obtain reproduction of the stereoscopic effect with a photo-couple: (1) A pair of images must be taken on the same object at slightly different angles; (2) The observer must be able to use his eyes separately in viewing the images at the same time, i.e. to make each eye see only the corresponding image separately and simultaneously; (3) The photo-couple must be set up in a definitive orientation, i.e. when viewing with both eyes, the two lines of sight from the corresponding points of the photo-couple must intersect. The presently discussed apparatus are designed to fulfill these conditions.

A further scrutiny on the general formulas derived above for the coordinates of feature points in space suggests that one has to measure the parallax differences of the corresponding point images. Hence, the following conditions must be further fulfilled in the design and construction of a quantitative stereoscopic radiography instrument: (4) There must be a device or a pair of devices to display a pair of images; (5) Two distinct sets of optical systems (preferably with some magnifying capability) may be advantageously used (also not a requirement) to facilitate the viewing by each eye of the respective image independently and simultaneously; (6) Adjustments must be allowed for the X- and Y-directional displacements for the image display devices and the eyepieces so that point images in various parts of the image can be seen. (7) The two images must be allowed to shift horizontally with respect to each other and there must be some devices for displacement measurements; (8) Reference lines and markers must be supplied for stereoscopic surveying. The presently discussed apparatus have fully met the above-cited requirements.

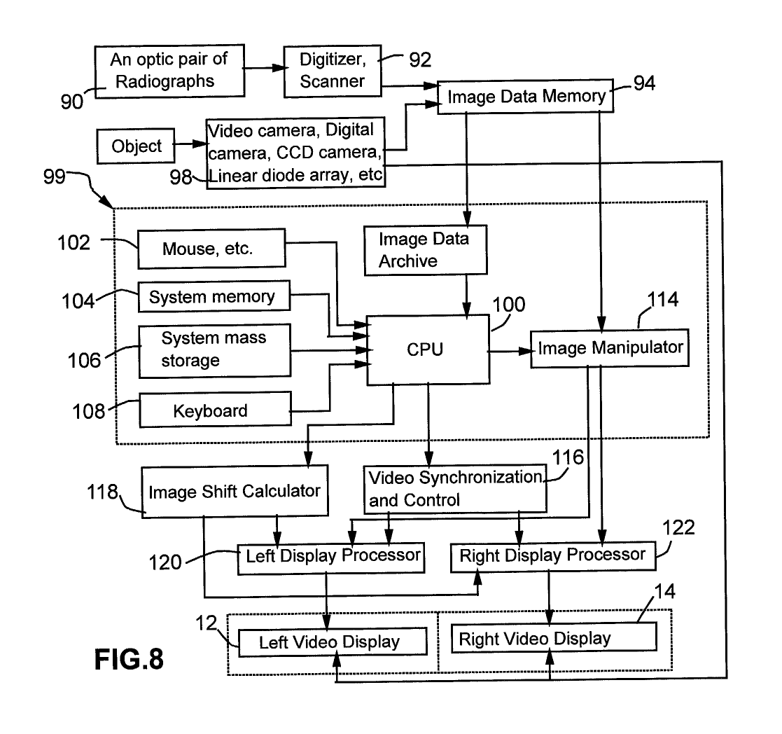

The nature of the image display devices is further discussed herein. In its simplest form, the image plate may be just a radiographic film (negative film or transparency) or a positive print (opaque photographic paper). In the case of radiographic transparencies, a pair of film boxes with back illuminating light constitute the two required display devices. When positive prints are employed, the two display devices are simply some devices that are capable of holding a pair of prints on their flat front surfaces. When deemed necessary, the front surfaces may be illuminated with proper lighting to facilitate observation. Alternatively, referring to FIG. 8, the images in radiographs (90, negative or positive) may be stored in an image data memory 94 through a commonly used scanner or digitizer 92 for further uses later.

In fluoroscopy radiography, the images picked up by an image intensifier 96 (or any type of radiation sensor plate) may be recorded by a camera means 98, or other type of image sensor/reader, and stored in the image data memory 94. Image sensor means may include a fluorescence screen, a phosphor screen, an amorphous selenium plate, an amorphous silicon plate, a laser beam scanner, and combinations thereof. Memory 94 could be either an independent memory unit or a part of the mass storage 106 of a computer 99. The system computer 99 includes a central processing unit (CPU) 100, system memory 104, system mass storage devices 106, a keyboard 108, and a screen location selection device (e.g., a mouse 102). The mass storage devices 106 may include floppy disk drives and hard disk drives for storing an operating system. These storage devices 106 also store application programs for the system computer 99 and routines for manipulating the images shown on the image display devices 12,14 and for communicating with imaging devices such as a scanner or digitizer 92, image intensifier 96, or image data memory 94.

In one embodiment of the present invention, image manipulating routines are used to drive devices such as an image manipulator 114, image shift calculator 118, video synchronization and control 116, and video display processors 120,122. Many commercially available image processing packages contain the above image manipulating and calculating capabilities. This mix of devices 114,116,118,120,122 provide capabilities of shifting the pair of images (photo-couple) horizontally together and with respect to each other, and computing the various image shift distances required in the calculation of the coordinates of an internal flaw. In another embodiment, the two images can be shown on the screen of an image display device; only one image display device is required. These two images can be shifted together as well as shifted with respect to each other as desired. In this case, the two reference wires 16,18 will be preferentially placed near the middle of the left portion and the middle of the right portion of the screen, respectively. The two references 16,18 can be just two internally generated or externally drawn straight lines that will remain stationary when the images are being shifted. In a further preferred embodiment, the image analysis software has an image or pattern recognition program. This program could allow the second reference line to be automatically relocated to coincide with the second image point of a feature on a second image (e.g., the right image) once the first line is positioned to coincide with the first image point of the same feature on a first image (e.g., the left image). During such an image recognition and shifting procedure, the relative shift distance may be automatically computed and recorded.

In yet another embodiment in which a minimal image manipulating capability is needed, the sole purpose of this capability is to deliver the images to their respective image display devices 12,14. Additional image enhancing functions to improve the image quality (resolution, contrast, etc.) are nice features to have, but are not strictly required. The movements of these images are to be executed by the primary platform 30 and secondary platform 28. In still another embodiment, at least one of the two image display devices has the capability of shifting the image horizontally with reference to the other image so that the secondary platform 28 (in FIG. 1(A)) can be eliminated. In this situation, the two image display devices 12,14 are both held in place by the primary platform 30, which provides simultaneous horizontal movements of the two display devices. The two display devices are maintained at a constant separation at all times.

It is to be understood that while certain forms of the present invention have been illustrated and described herein, the invention is not to be limited to the specific forms or arrangement of the parts described and shown.

What is claimed:

1. A quantitative radiographic method of determining the depth of a selected feature inside a three-dimensional object from a stereoscopic pair of left and right radiographic images to be presented to the left eye and right eye, respectively, of an observer; said method comprising the steps of:
(a) producing said pair of radiographic images on the same object at slightly different angles, comprising:
i. placing a high-energy radiation source to one side of said object and a planar radiation sensor means to an opposite or back side of said object so that said radiation source, object and sensor means are aligned in a substantially straight line;
ii. defining an X-Y-Z rectangular coordinate system in which the direction from the geometric center of said film to said radiation source approximately defines the Z-axis, the width direction of said planar sensor means being also substantially parallel to the line segment connecting the two eyes of said observer defines the horizontal X-axis direction, and a third axis perpendicular to both X- and Z-axes defines the transverse Y-axis direction;
iii. providing a reference marker G at a selected position on or near the front surface of said object facing said radiation source so that the image of said marker can be detected by said radiation sensor means for the purpose of serving as an image reference point;
iv. generating the left image by irradiating a high energy radiation beam from said radiation source through said object and finally reaching said sensor means with said marker G forming an image point g₁ in said left image;
v. generating the right image by resetting said sensor means, effecting a horizontal shift of said radiation source along the X-axis direction with respect to said sensor means by a small displacement B or by tilting said radiation source around the Y-axis by a small angle inclined with respect to the Z-axis, and exposing said sensor means to a radiation beam from said radiation source under a substantially identical exposure condition with said marker G leaving an image point g₂ in said right image;
(b) using image display means to present said left image to the left eye of said observer and said right image to the right eye of said observer so that the two images can be observed by the left and right eyes separately; said two images being set up in a definitive orientation so that the line segment connecting the two eyes of said observer is substantially parallel to the X-axis; said two images being provided with two stationary, transversely aligned reference lines, referred to as the left reference line and right reference line, respectively, across the image plane in the Y-direction and lying substantially on or very close to said image plane; the two images being substantially at the same Y-axis position; and
(c) performing and measuring horizontal shifting motions of said two images and obtaining the depth coordinate, Z_{GA}, of an internal feature A with respect to marker G according to the following procedures:
i. Shift the two images in the X-direction until the right image point g₂ of marker G on the right image falls on the right reference line and the corresponding image point g₁ of said marker G on the left image falls on the left reference line;
ii During or after the shifting procedure (c)-i, use displacement-metering means to measure and record a travel distance P_{G} of the left image relative to the right image;
iii Shift said two images in the X-direction to bring an image point a₂ of an internal feature A of interest on said right image to fall on said right reference line and the corresponding image point a₁ of said feature A on said left image to fall on said left reference line;
iv During or after the shifting procedure, measure and record the travel distance P_{A} of the left image relative to the right image to obtain a relative image shift quantity defined as ΔP_{GA}=P_{G}−P_{A}; and
v Use the formula Z_{GA}=(H/B)ΔP_{GA} to calculate the vertical depth or Z-coordinate, Z_{GA}, of said feature A with respect to said marker G, where H is the vertical distance from said radiation source to said front surface of the object.

2. The method as set forth in claim 1 in which said two images are recorded in the form of a film, positive photographic print, video image on a video display device, or digital image on a computer monitor.

3. The method of claim 1, comprising the further steps of using displacement-metering means to measure the X-directional separation ΔXga between the image point g of said marker G and the image point a₁ of said feature A on said left image, defining F to be the vertical focal length between said radiation source and said radiation sensor means while being exposed to said radiation beam, and then using the following formula to calculate the X-coordinate of said feature A:
$${X}_{G A} = \frac{B}{2} - \frac{( H + {Z}_{G A} ) ( \frac{F \cdot B}{2} - H \cdot Δ {X}_{g a} )}{F \cdot H} .$$

4. The method of claim 1, comprising the further steps of measuring the Y-directional separation ΔYga between said image point g of G and said image point a₁ of A on said left image, drawing an imaginary vertical line from said radiation source to said planar sensor means while being exposed to said radiation beam, defining and measuring Y_{G} to be the Y-directional separation between G and said imaginary vertical line, and using the following formula to calculate the Y-coordinate of said feature A:
$${Y}_{G A} = \frac{Δ {Y}_{ga} ( H + {Z}_{G A} )}{F} - \frac{{Y}_{G} \cdot {Z}_{G A}}{H} .$$

5. A method as set forth in claim 1 including the additional steps of
(a) providing another marker K on or near the back surface of said object to produce its corresponding image points k₁ and k₂ in said left image and right image, respectively;
(b) performing and measuring horizontal shifting motions of said two images according to the following additional procedures:
i. Shift said two images independently or simultaneously in the X-direction to bring the image point k₂ on said right image to fall on the right reference line and bring the corresponding image k₁ on said left image to fall on the left reference line; and
ii. record the travel distance of the left image with respect to the right image as Pₖ, and then obtain a second image shift quantity defined as ΔP_{Gk}=P_{G}−Pₖ;
(c) Obtain more accurate H values by using the following formulas, H=h B/ΔP_{Gk} and then follow the procedures specified in (c)-v of claim 1 to obtain more accurate values of Z_{GA}=h ΔP_{GA}/ΔP_{Gk}.

6. A method as set forth in claim 3 including the additional steps of
(a) providing another marker K on or near the back surface of said object to produce its corresponding image points k₁ and k₂ in said left image and right image, respectively;
(b) performing and measuring horizontal shifting motions of said two images according to the following additional procedures:
i. Shift said two images independently or simultaneously in the X-direction to bring the image point k₂ on said right image to fall on the right reference line and bring the corresponding image k₁ on said left image to fall on the left reference line; and
ii. record the travel distance of the left image with respect to the right image as Pₖ, and then obtain a second image shift quantity defined as ΔP_{Gk}=P_{G}−Pₖ;
(c) Obtain more accurate F and H values by using the following formulas: H=h B/ΔP_{Gk} and F=h(1+B/ΔP_{Gk}), and then use said more accurate F and H values to calculate more accurate values of X_{GA}.

7. A method as set forth in claim 4 including the additional steps of
(a) providing another marker K on or near the back surface of said object to produce its corresponding image points k₁ and k₂ in said left image and right image, respectively;
(b) performing and measuring horizontal shifting motions of said two images according to the following additional procedures:
i. Shift said two images independently or simultaneously in the X-direction to bring the image point k₂ on said right image to fall on the right reference line and bring the corresponding image k₁ on said left image to fall on the left reference line; and
ii. record the travel distance of the left image with respect to the right image as Pₖ, and then obtain a second image shift quantity defined as ΔP_{Gk}=P_{G}−Pₖ;
(c) Obtain more accurate F and H values by using the following formulas: H=h B/ΔP_{Gk} and F=h (1+B/ΔP_{Gk}), and then use said more accurate F and H values to calculate more accurate values of Y_{GA}.

8. The method of claim 1, further comprising additional step of operating a stereoscope means for viewing said left and right images.

9. The method of claim 1, wherein said left image and right image being digital images displayed on a computer monitor and said left and right reference lines being either internally generated and displayed on said monitor or written on said monitor with a marking pen.

10. The method of claim 1, wherein said left and right reference lines being thin wires or filaments.

11. The method of claim 1, wherein said radiation being selected from the group consisting of X-ray, Gamma ray, or neutron radiation.

12. The method of claim 1, wherein said radiation sensor means comprising an unexposed radiographic film, an image intensifier, a fluorescence screen, a phosphor screen, an amorphous selenium plate, an amorphous silicon plate, a laser beam scanner, and combinations thereof.

13. The method of claim 1, wherein said reference marker G being selected from a feature of said object with a known location.

14. The method of claim 1, wherein said displacement-metering means comprising monitor pixel-counting means effected by operating a keyboard, a mouse, a joystick, or combinations thereof.

15. The method of claim 1, wherein said image display means comprising a film box supported by a linear motion device and said displacement-metering means comprising a displacement sensor mechanically, optically, and/or electronically connected to said film box or said linear motion device.

16. The method of claim 5, wherein said reference marker K being selected from a feature of said object with a known location.

17. The method of claim 1, wherein said steps of performing and measuring horizontal shifting motions of said two images comprising operating a pattern recognition program in a computer in such a fashion that one or both of said left image and right image can be shifted automatically so that a desired feature of said object or a marker coincides with at least one of said two reference lines.

18. The method of claim 5, wherein said steps of performing and measuring horizontal shifting motions of said two images comprising operating a pattern recognition program in a computer in such a fashion that one or both of said left image and right image can be shifted automatically on a monitor so that a desired feature of said object or a marker coincides with at least one of said two reference lines.

19. The method of claim 1, wherein said step of resetting said sensor means comprising replacing an exposed film with an un-exposed film.

20. An apparatus for stereoscopically displaying a pair of left and right radiographic images that are taken from slightly different angles of an object and for determining the spatial coordinates of a selected feature image inside said object, comprising:
(a) two parallel image display devices, a left one for presenting said left image to the left eye and a right one for presenting said right image to the right eye of an observer; said two images being placed side-by-side along an X-axis direction of an X-Y-Z rectangular coordinate system, said X-axis being defined to be along a width direction of said images and lying approximately on a plane containing said images as well as being substantially parallel to the line segment connecting the two eyes of said observer; the Y-axis of said coordinate system being along the length direction of said images, perpendicular to the X-axis direction, and also lying approximately on said image plane with the Z-axis being normal to said image plane;
(b) a left secondary platform to support said left image display device and a right secondary platform to support said right image display device; said left and right secondary platforms being provided with movement means to reversibly displace said two display devices with respect to each other horizontally in the X-direction; said movement means being equipped with displacement-measuring means to measure out a relative shift distance between said two display devices;
(c) a sturdy base in close proximity to support said secondary platforms;
(d) a stereoscope-type observing device in working proximity to said image display devices, comprising two parallel optical paths with each optical path comprising reflector means to direct said left image into the left eye and said right image into the right eye of the observer; said optical paths being housed and protected by a casing means which is connected to a supporting member; said supporting member being provided with drive means to reversibly move said optical paths transversely in the Y-direction; said supporting member being further supported by a sturdy base; and
(f) two parallel reference lines transversely aligned in the Y-direction, a left reference line lying across a front end of said left optical path proximal to said left image and a right reference line lying across a front end of said right optical path proximal to said right image; said reference lines being held in place on said casing means by a fastening means.

21. The apparatus as set forth in claim 20 wherein said image display devices are video display monitors.

22. The apparatus as set forth in claim 20 wherein said image display devices are video display monitors which are in electronic communication with the following image acquiring and processing devices:
(a) image recording means to acquire images from a radiographic film, image intensifier, fluorescence screen, phosphor screen, amorphous selenium plate, amorphous silicon plate, laser beam scanner, or combinations thereof; and
(b) a computer for image storing and processing, said computer being in electronic communication with said image recording means and comprising a system memory, system mass storage, a keyboard, a screen location-selecting device, and image manipulator and processor means.

23. The apparatus as set forth in claim 20 wherein each said image display device is a radiographic film supporting and illuminating means comprising a generally rectangular casing, an optically transparent plate attached to said rectangular casing to support a flat radiographic film, clip means to hold said film against a surface of said transparent plate, and a light source behind said transparent plate and inside said rectangular casing to illuminate said film.

24. The apparatus as set forth in claim 20 wherein said two parallel reference lines are two thin wires transversely aligned in the Y-direction, the left reference line lying across the front surface of said left image display device and the right reference line across the front surface of said right image display device; said reference lines being held in place by said sturdy base of the platforms so that said reference lines remain stationary while said secondary platforms are in motion.

25. The apparatus as set forth in claim 20 wherein said platform movement means are provided with
(a) displacement sensor means to convert displacement data into a digital form;
(b) electronic calculator means in electronic communication with said displacement sensor means to calculate image shift distances and the spatial location of a selected internal feature of said object; and
(c) digital display means in electronic communication with said calculator means to show the calculated data values as desired.
